SEQUENCE LISTING (26 sequences)

SEQ ID NO: 1 - protein, 40 aa, Artificial Sequence
  Synthetic NTD domain
MSNNNYSNGLNPDESLSASAFDPNLVGPTLPPIPPFTLPT

SEQ ID NO: 2 - DNA, 120 bp, Artificial Sequence
  Synthetic NTD domain
atgtcaaataataattattcaaatggattaaaccccgatgaatctttatcagctagtgcatttgaccctaatcttgtaggacctacattaccaccgataccaccatttacccttcctacc

SEQ ID NO: 3 - protein, 115 aa, Artificial Sequence
  Synthetic BclA NTD-CLR domain
MSNNNYSNGLNPDESLSASAFDPNLVGPTLPPIPPFTLPTGPTGPTGPTGPTGPTGPTGPTGPTGPTGPTGPTGPTGPTGPTGDTGTTGPTGPTGPTGPTGPTGATGLTGPTGPT

SEQ ID NO: 4 - DNA, 345 bp, Artificial Sequence
  Synthetic BclA NTD-CLR domain gene
atgtcaaataataattattcaaatggattaaaccccgatgaatctttatcagctagtgcatttgaccctaatcttgtaggacctacattaccaccgataccaccatttacccttcctaccggaccaactgggccgactggaccgactgggccgactgggccaactggaccaactgggccgactgggccaactggaccaactggaccaactgggccaactggaccaactgggccaactgggccaactggagacactggtactactggaccaactgggccaactggaccaactggaccaactgggccaactggtgctaccggactgactggaccgactggaccgact

SEQ ID NO: 5 - protein, 253 aa, Bacillus anthracis
MSNNNYSNGLNPDESLSASAFDPNLVGPTLPPIPPFTLPTGPTGPTGPTGPTGPTGPTGPTGPTGPTGPTGPTGPTGPTGPTGDTGTTGPTGPTGPTGPTGPTGATGLTGPTGPTGPSGLGLPAGLYAFNSGGISLDLGINDPVPFNTVGSQFGTAISQLDADTFVISETGFYKITVIANTATASVLGGLTIQVNGVPVPGTGSSLISLGAPIVIQAITQITTTPSLVEVIVTGLGLSLALGTSASIIIEKVA

SEQ ID NO: 6 - DNA, 762 bp, Bacillus anthracis
atgtcaaataataattattcaaatggattaaaccccgatgaatctttatcagctagtgcatttgaccctaatcttgtaggacctacattaccaccgataccaccatttacccttcctaccggaccaactgggccgactggaccgactgggccgactgggccaactggaccaactgggccgactgggccaactggaccaactggaccaactgggccaactggaccaactgggccaactgggccaactggagacactggtactactggaccaactgggccaactggaccaactggaccaactgggccaactggtgctaccggactgactggaccgactggaccgactgggccatccggactaggacttccagcaggactatatgcatttaactccggtgggatttctttagatttaggaattaatgatccagtaccatttaatactgttggatctcagtttggtacagcaatttctcaattagatgctgatactttcgtaattagtgaaactggattctataaaattactgttatcgctaatactgcaacagcaagtgtattaggaggtcttacaatccaagtgaatggagtacctgtaccaggtactggatcaagtttgatttcactcggagcacctatcgttattcaagcaattacgcaaattacgacaactccatcattagttgaagtaattgttacagggcttggactatcactagctcttggcacgagtgcatccattattattgaaaaagttgcttaa

SEQ ID NO: 7 - protein, 304 aa, Burkholderia
  misc_feature (1)..(304): sp. FA1
MFEGFERRLVDVGDVTINCVVGGSGPALLLLHGFPQNLHMWARVAPLLANEYTVVCADLRGYGGSSKPVGAPDHANYSFRAMASDQRELMRTLGFERFHLVGHDRGGRTGHRMALDHPDSVLSLAVLDIIPTYVMFEEVDRFVARAYWHWYFLQQPAPYPEKVIGADPDTFYEGCLFGWGATGADGFDPEQLEEYRKQWRDPAAIHGSCCDYRAGGTIDFELDHGDLGRQVQCPALVFSGSAGLMHSLFEMQVVWAPRLANMRFASLPGGHFFVDRFPDDTARILREFLSDARSGIHQTERRES

SEQ ID NO: 8 - DNA, 915 bp, Burkholderia
  misc_feature (1)..(915): sp. FA1
atgttcgagggtttcgagcgtcgtctggtcgacgtaggtgacgtcactatcaactgcgtcgtgggcggttctggtcctgcactgctgctgctgcatggtttccctcagaacctgcacatgtgggcacgtgtggcaccactgctggcaaacgagtacactgtggtgtgcgctgacctgcgtggttacggtggttcttctaagccagtgggtgcaccagaccatgcaaactactccttccgcgctatggcctctgaccagcgtgaactgatgcgcactctgggtttcgaacgtttccacctggtgggtcacgaccgtggtggtcgtaccggtcatcgtatggctctggaccatccggattctgttctgtccctggctgttctggacatcatcccgacctacgttatgttcgaagaagttgaccgcttcgttgcgcgtgcctactggcactggtacttcctgcagcagccggcgccgtacccggagaaagttatcggcgcggacccggataccttctatgaaggctgcctgttcggctggggtgctacgggtgctgatggtttcgacccggaacaactggaagaatatcgtaaacagtggcgcgacccggctgctatccacggctcttgctgtgattatcgtgcgggcggcaccattgatttcgaactggatcacggcgatctgggccgtcaggttcagtgtccggcgctggtattttccggcagcgccggcctgatgcactccctgtttgaaatgcaggttgtatgggcgccgcgtctggcgaatatgcgttttgcgtccctgccgggcggccactttttcgtagatcgttttccggatgataccgcccgcattctgcgcgaatttctgagcgatgcgcgcagcggcattcaccaaaccgaacgccgcgaaagctaa

SEQ ID NO: 9 - protein, 236 aa, Bacillus bombysepticus
  misc_feature (1)..(236): SF3
MKYKFILFDVDDTLLDFPETERHALHNAFVQFGMPTGYNDYLASYKEISNGLWRDLENKMITLSELAVDRFRQLFALHNIKVDAQQFSDVYLENLGKEVHLIEGAVQLCEDLQDCKLGIITNGYTKVQQSRIGNSPVCNFFDHIIISEEVGHQKPAREIFDYAFEKFGITDKSSVLMVGDSLSSDMRGGEDYGIDTCWYNPSLKENRTDVKPSYEVESLLQILEIVEVTKEKVASF

SEQ ID NO: 10 - DNA, 711 bp, Bacillus bombysepticus
  misc_feature (1)..(711): SF3
atgaaatacaaatttatattattcgacgtagacgatacattattagatttccctgaaacggaaagacacgcattacataatgcgtttgtacagttcgggatgcctacagggtataatgattatcttgcaagttataaagagattagtaatggattatggagagatttagaaaataaaatgattacgctaagtgaattagcggtagatcgatttagacaattatttgcccttcataatataaaagtagatgcgcagcaatttagcgatgtatatcttgaaaacttagggaaagaagtacatcttatagaaggtgcagtgcaattatgtgaggatctacaagattgcaagttaggtattattacgaatggatatacgaaggtgcaacaatcgagaattggaaattcgcctgtatgtaatttctttgatcatattattatttcagaagaggttggtcatcaaaaaccagcacgtgagatttttgattatgcgtttgaaaagtttgggattacagataaatcaagtgtattaatggttggagattcgctttcttctgatatgagaggcggagaagattacggcattgatacgtgttggtataatccgagtttgaaagaaaataggacagatgttaagccgtcttatgaagtggagagtctgctacaaattttagaaattgtagaagtgactaaagaaaaagtagcttcattttaa

SEQ ID NO: 11 - protein, 253 aa, Bacillus anthracis; identical to SEQ ID NO: 5

SEQ ID NO: 12 - DNA, 762 bp, Bacillus anthracis; identical to SEQ ID NO: 6

SEQ ID NO: 13 - protein, 21 aa, Artificial Sequence
  Synthetic BAN domain
AFDPNLVGPTLPPIPPFTLPT

SEQ ID NO: 14 - DNA, 63 bp, Artificial Sequence
  Synthetic BAN domain gene
gcattcgacccgaatcttgtgggtccgaccttgccgcctattccaccatttaccctgccaacc

SEQ ID NO: 15 - protein, 40 aa, Artificial Sequence; identical to SEQ ID NO: 1
  Synthetic BANF domain

SEQ ID NO: 16 - DNA, 120 bp, Artificial Sequence
  Synthetic BANF domain gene
atgagtaacaataattatagtaatggcttaaatccggacgaaagtttatctgcatcagcattcgacccgaatcttgtgggtccgaccttgccgcctattccaccatttaccctgccaacc

SEQ ID NO: 17 - DNA, 38 bp, Artificial Sequence
  Synthetic primer
cacacaggaaacagaccatgaaatacaaatttatatta

SEQ ID NO: 18 - DNA, 39 bp, Artificial Sequence
  Synthetic primer
cctgcaggtcgactctagttaaaatgaagctactttttc

SEQ ID NO: 19 - DNA, 37 bp, Artificial Sequence
  Synthetic primer
cacacaggaaacagaccatgagtaacaataattatag

SEQ ID NO: 20 - DNA, 39 bp, Artificial Sequence
  Synthetic primer
atataaatttgtatttcatggttggcagggtaaatggtg

SEQ ID NO: 21 - DNA, 39 bp, Artificial Sequence
  Synthetic primer
caccatttaccctgccaaccatgaaatacaaatttatat

SEQ ID NO: 22 - DNA, 38 bp, Artificial Sequence
  Synthetic primer
cacacaggaaacagaccatgttcgagggtttcgagcgt

SEQ ID NO: 23 - DNA, 39 bp, Artificial Sequence
  Synthetic primer
tgcctgcaggtcgactctagttagctttcgcggcgttcg

SEQ ID NO: 24 - DNA, 39 bp, Artificial Sequence
  Synthetic primer
gctcgaaaccctcgaacatggttggcagggtaaatggtg

SEQ ID NO: 25 - DNA, 39 bp, Artificial Sequence
  Synthetic primer
caccatttaccctgccaaccatgttcgagggtttcgagc

SEQ ID NO: 26 - DNA, 36 bp, Artificial Sequence
  Synthetic primer
cacacaggaaacagaccatggcattcgacccgaatc

(19) United States
(12) Patent Application Publication
(10) Pub. No.: US 2019/0144871 A1
(43) Pub. Date: May 16, 2019

(54) POLYNUCLEOTIDE ENCODING FUSION OF ANCHORING MOTIF AND DEHALOGENASE, HOST CELL INCLUDING THE POLYNUCLEOTIDE, AND USE THEREOF

(71) Applicant: Samsung Electronics Co., Ltd., Suwon-si (KR)

(72) Inventors: Yukyung Jung, Hwaseong-si (KR); Jongwon Byun, Suwon -si (KR); Soojin Park, Hwaseong-si (KR); Dongsik Yang, Seoul (KR); Jinhwan Park, Suwon -si (KR)

(21) Appl. No.: 16/047,404

(22) Filed: Jul. 27, 2018

(30) Foreign Application Priority Data
Nov. 14, 2017 (KR) 10-2017-0151719

(51) Int. Cl. C12N 15/62; C12N 15/11

(52) U.S. Cl. CPC C12N 15/62; C12N 15/111; C12Y 308/01003; C07K 2319/035; C07K 2319/02; C07K 2319/03; C07K 2319/33; C12Y 308/01002

(57) ABSTRACT

Provided is a linked polynucleotide in which polynucleotides encoding a promoter, an anchoring motif, and a dehalogenase, respectively, are operatively linked to one another; a host cell including the linked polynucleotide; and a method of reducing a concentration of a fluorine-containing compound in a sample by using the host cell.

### CROSS-REFERENCE TO RELATED APPLICATION

This application claims the benefit of Korean Patent Application No. 10-2017-0151719, filed on Nov. 14, 2017, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 19,486 Byte ASCII (Text) file named “739015_ST25.TXT,” created on Jul. 23, 2018.

### BACKGROUND

### 1. Field

The present disclosure relates to a linked polynucleotide including polynucleotides encoding a promoter, an anchoring motif, and a dehalogenase operatively linked to each other, a host cell including the linked polynucleotide, and a method of reducing a concentration of a fluorine-containing compound in a sample by using the host cell.

### 2. Description of the Related Art

A variety of halogenated compounds have been synthesized and used in commercial applications. Halogenated compounds are used in herbicides, pesticides, plastics, and solvents. They are also included in waste from electronics industries, such as semiconductors. Of the various halogenated compounds, fluorine-containing compounds have a long half-life and a considerably high global warming potential, thus causing severe environmental contamination. Such fluorine-containing compounds may include perfluorocarbons (PFCs), hydrofluorocarbons (HFCs), or sulfur hexafluorides (SF₆).

A halogenated compound may be removed or converted into a non-toxic material through a pyrolysis or catalytic thermal oxidation process, but it still has disadvantages, such as a limited decomposition rate, emission of secondary harmful materials, and high cost.

Therefore, there is a need to develop new microorganisms that reduce a halogenated compound in a sample.

### SUMMARY

Provided herein is a linked polynucleotide comprising a first polynucleotide, a second polynucleotide, and a third polynucleotide that are operatively linked to one another, wherein the first polynucleotide is a promoter polynucleotide, the second polynucleotide encodes an anchoring motif, and the third polynucleotide encodes a dehalogenase.

Also provided is a host cell comprising the linked polynucleotide.

Further provided is a method of reducing the concentration of a fluorine-containing compound in a sample by contacting the sample with the host cell.

Additional aspects of the invention will be set forth, in part, in the description that follows, and will be apparent from the description, or may be learned by practice of the presented embodiments.

### BRIEF DESCRIPTION OF THE DRAWINGS

These and/or other aspects will become apparent and more readily appreciated from the following description of the embodiments, taken in conjunction with the accompanying drawings in which:

FIG. 1A is a vector map of the vector pTrc-SF0757;

FIG. 1B is a vector map of the vector pTrc-BANF-SF0757;

FIG. 2 is a schematic view of a glass Dimroth coiled reflux condenser;

FIG. 3A is a vector map of the pTrc-FAcD vector; and

FIG. 3B is a vector map of the pTrc-BANF-FAcD vector.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

The term “gene” as used herein may refer to a polynucleotide that encodes a particular protein. A gene may optionally include at least one regulatory sequence of a 5′-non-coding sequence and a 3′-non-coding sequence, or may be free from regulatory sequences.

The term “sequence identity” of a polynucleotide (nucleic acid) or polypeptide as used herein refers to a degree of identity between bases or amino acid residues of two corresponding sequences over a particular region. The sequence identity is a value that is measured by comparing two optimally aligned corresponding sequences of a particular comparable region. In some embodiments, a percentage of the sequence identity may be calculated by comparing two optimally aligned corresponding sequences in an entire comparable region, determining the number of locations where the two sequences have an identical amino acid or an identical nucleic acid to obtain the number of matched locations, dividing the number of the matched locations by the total number (that is, a range size) of all locations within a comparable range, and multiplying the result by 100 to obtain a percentage of the sequence identity. The percentage of the sequence identity may be determined by using known sequence comparison programs, examples of which include BLASTN (NCBI) and BLASTP (NCBI), CLC Main Workbench (CLC bio.), and MegAlign™ (DNASTAR Inc).

A “fluorine-containing compound” as used herein means a compound of Formula 1, Formula 2, or Formula 3:

C(R₁)(R₂)(R₃)(R₄) <Formula 1>

(R₅)(R₆)(R₇)C—[C(R₁₁)(R₁₂)]n-C(R₈)(R₉)(R₁₀) <Formula 2>

(R₁₃)(R₁₄)(R₁₅)C—[C(R₁₉)(R₂₀)]n-A. <Formula 3>

In Formula 1, 2, and 3, n may be an integer from 0 to 10.

When n in Formula 2 is 2 or greater, each of Ru may be the same as or different from one another, and each of R₁₂ may be the same as or different from one another.

When n in Formula 3 is 2 or greater, each of R₁₉ may be the same as or different from one another, and each of R₂₀ may be the same as or different from one another.

A may be —COOH or —C(R₁₆)(R₁₇)(R₁₈).

R₁, R₂, R₃, and R₄ may each independently be F, Cl, Br, I, or H. In one embodiment, at least one of R₁, R₂, R₃, and R₄ may be F.

R₅, R₆, R₇, R₈, R₉, R₁₀, R₁₁, and R₁₂ may each independently be F, Cl, Br, I, or H. In one embodiment, at least one of R₅, R₆, R₇, R₈, R₉, R₁₀, R₁₁, and R₁₂ may be F.

R₁₃, R₁₄, R₁₅, R₁₆, R₁₇, R₁₈, R₁₉, and R₂₀ may each independently be F, Cl, Br, I, H, or —COOH. In one embodiment, at least one of R₁₃, R₁₄, R₁₅, R₁₆, R₁₇, R₁₈, R₁₉, and R₂₀ may be F. In a further embodiment, at least one of R₁₃, R₁₄, R₁₅, R₁₆, R₁₇, R₁₈, R₁₉, and R₂₀ may be —COOH. Examples of fluorine-containing compounds include CH₃F, CH₂F₂, CHF₃, CF₄, CH₂FCOOH, and mixtures thereof.

In accordance with an aspect of the disclosure, a linked polynucleotide includes a first polynucleotide, a second polynucleotide, and a third polynucleotide that are operatively linked to one another, wherein the first polynucleotide is a promoter polynucleotide, the second polynucleotide is a polynucleotide encoding an anchoring motif, and the third polynucleotide is a polynucleotide encoding a dehalogenase.

The linked polynucleotide may be operatively linked to additional non-coding and/or expression regulatory sequences required for gene expression in a host cell, if necessary. For instance, the linked polynucleotide may include an origin of replication, a promoter, a cloning site, a marker, or a combination thereof.

The linked polynucleotide may be introduced into a host cell. The introduction may be implemented using a known method such as electroporation, electric shock, transformation, or transduction. The linked polynucleotide may be part of a nucleic acid construct that enables gene expression in a host cell, such as a vector or a plasmid.

The promoter of the linked polynucleotide may be homologous or heterologous to the host cell. The promoter is not particularly limited, and selection of the promoter may depend in part on the particular host cell in which the linked polynucleotide will be used. Examples of promoters include, for example, a Trc, Tac, araBAD, or T5 promoter.

The linked polynucleotide is constructed to encode a fusion protein comprising the anchoring motif and the dehalogenase, such that, when introduced into a host cell, the cell will express the fusion protein of the anchoring motif and the dehalogenase. The anchoring motif anchors the fusion protein in the cell membrane and dehalogenase is expressed on the surface of the host cell.

The anchoring motif may be any molecule that enables the protein to which it fuses to adhere to the cell surface. The anchoring motif may include a transmembrane portion and a linker portion, wherein the linker portion is connected with the transmembrane portion so as to be positioned in a direction away from the cell surface. In other words, the linker portion is orientated away from the interior of the cell. The linker portion, when present, links and is between the transmembrane portion and the dehalogenase.

The anchoring motif may be provided by, for instance, a membrane protein, a lipoprotein, an autotransporter protein, or any transmembrane domain. By way of illustration, the anchoring motif may be selected from the group consisting of BclA of Bacillus; OmpA, Lpp-OmpA, OmpC, OmpS, LamB, OmpC, Lpp-OmpC, PhoE, and FadL of Escherichia coli (E. coli); OmpC of Salmonella; OprF of Pseudomonas; adhesin involved in diffusion adherence (AIDA-I) of pathogenic E. coli, and fragments thereof (e.g., transmembrane domains thereof). The linker portion may consist of about 15 to 200 amino acids, about 15 to 150 amino acids, about 15 to 100 amino acids, about 15 to 80 amino acids, about 15 to 60 amino acids, about 15 to 50 amino acids, or about 15 to 40 amino acids.

In some embodiments, the anchoring motif may be BclA having an amino acid sequence of SEQ ID NO: 1 (NTD sequence), SEQ ID NO: 3 (NTD-CLR sequence), SEQ ID NO: 5 (NTD-CLR-CTD sequence), or fragments thereof (e.g., transmembrane domain thereof). The amino acid sequences of SEQ ID NOs: 1, 3, and 5 may be encoded by the nucleotide sequences of SEQ ID NOs: 2, 4, and 6, respectively.

The term “dehalogenase” as used herein may refer to an enzyme that catalyzes the removal of a halogen atom from a substrate or the conversion of a halogen-containing substrate into another compound. The dehalogenase may be 4-chlorobenzoate dehalogenase, 4-chlorobenzoyl-CoA dehalogenase, dichloromethane dehalogenase, fluoroacetate (FA) dehalogenase, haloacetate dehalogenase, (R)-2-haloacid dehalogenase, (S)-2-haloacid dehalogenase, haloalkane dehalogenase, halohydrin dehalogenase, or tetrachloroethene reductive dehalogenase. For example, the dehalogenase may belong to a haloacid dehalogenase superfamily. The haloacid dehalogenase superfamily may be EC 3.8.1.2. However, the present disclosure should not be construed as being limited to this particular mechanism. The dehalogenase may have a sequence identity of about 85% or greater, 90% or greater, 95% or greater, 96% or greater, 97% or greater, 98% or greater, or 99% or greater with an amino acid sequence of SEQ ID NO: 7 (FAcD amino acid sequence) or SEQ ID NO: 9 (SF0757 amino acid sequence). The fluoroacetate (FA) dehalogenase may belong to EC 3.8.1.3.

The linked polynucleotide may further include a fourth polynucleotide that encodes a signal polypeptide. The polynucleotide encoding the signal peptide may be operatively linked to the polynucleotide encoding the anchoring motif and/or dehalogenase. For example, the fourth polynucleotide may be linked between the first polynucleotide and the second polynucleotide. The signal polypeptide may allow a synthesized polypeptide to be embedded in or to pass through the cell membrane. In one embodiment, the signal polypeptide is removed from the mature polypeptide via cleavage. In another embodiment, the signal polypeptide remains in the mature polypeptide.

In accordance with another aspect of the disclosure, there is provided a host cell including the linked polynucleotide according to any of the embodiments described herein. The host cell expresses the linked polynucleotide, which encodes a fusion protein of the anchoring motif and the dehalogenase, and the dehalogenase is expressed on the surface of the cell. All aspects of the linked polynucleotide are as previously described.

The host cell may be a microbial cell, such as yeast or bacteria. The bacteria may belong to the genus Escherichia, Bacillus, Pseudomonas, or Xanthobacter. The yeast may belong to the genus Saccharomyces.

The host cell may further include a dehalogenase-encoding polynucleotide that is not linked to the polynucleotide encoding the anchoring motif, wherein the cell simultaneously expresses the dehalogenase on the cell surface and a dehalogenase that remains inside the cell (internally active dehalogenase). In one embodiment, the microorganism may inherently express the dehalogenase that remains in the cell. In another embodiment, the microorganism may be genetically modified to express the dehalogenase via a genetic modification that increases expression of a gene encoding the dehalogenase for example, by introducing an exogenous polynucleotide that encodes the dehalogenase that remains in the cell, which polynucleotide might be heterologous or endogenous to the cell. Thus, the microorganism may include a polynucleotide that encodes a first dehalogenase, and a polynucleotide that encodes a second dehalogenase, wherein the first dehalogenase is expressed only within the cell, and the second dehalogenase is expressed on the surface of the cell. The polynucleotide that encodes the first dehalogenase and the polynucleotide that encodes the second dehalogenase may both be introduced by genetic modification. The first dehalogenase and the second dehalogenase may be the same or different from one another.

Introduction of the linked polynucleotide into the host cell, in some embodiments, provides a host cell with enhanced ability to degrade fluorine-containing compounds in a sample with which the host cell is contacted as compared to the same host cell without the linked polynucleotide. Thus, in another aspect, the disclosure provides a method of providing a recombinant cell with enhanced ability to degrade a fluorine containing compound, the method comprising introducing the linked polynucleotide described herein to a host cell. The linked polynucleotide and host cell are as previously described herein with respect to those aspects of the disclosure.

In accordance with another aspect of the present invention, a method of reducing a concentration of a fluorine-containing compound in a sample includes contacting the sample comprising a fluorine-containing compound with a host cell comprising the linked polynucleotide according to any of the above-described embodiments to reduce the concentration of the fluorine-containing compound in the sample.

The host cell may be any of the embodiments described herein. Thus, the host cell expresses dehalogenase on the cell surface. The host cell may further include a dehalogenase-encoding polynucleotide that is not linked to the polynucleotide encoding the anchoring motif, such that the host cell simultaneously expresses a dehalogenase on the cell surface and expresses a dehalogenase that remains inside the cell.

The host cell may reduce a concentration of the “fluorine-containing compound” in a sample. In one embodiment, the fluorine-containing compound may be CH₃F, CH₂F₂, CHF₃, CF₄, CH₂FCOOH, or a mixture thereof.

In certain embodiments, the reduction of the concentration of the fluorine-containing compound may be achieved by cleavage of C—F bonds of the fluorine-containing compound by the polypeptide, by converting the fluorine-containing compound into another material, or by accumulation of the fluorine-containing compound in cells.

The sample comprising the fluorine-containing compound may be a liquid sample, a gaseous sample, or a combination thereof. In one embodiment, the sample does not include the host cell. The sample may be industrial sewage or waste gas. For example, the sample may be industrial sludge. The term “sludge” refers to a semi-solid slurry and can be produced as sewage sludge from wastewater treatment processes or as a settled suspension obtained from conventional drinking water treatment and numerous other industrial processes.

Contacting the sample with the host cell may be performed in a liquid phase, a gaseous phase, or a combination thereof. For example, a gaseous sample may be contacted with the host cell in a liquid phase at the interface or the surface of the liquid. The contacting may include culturing the host cell in the presence of the fluorine-containing compound or sample comprising same. The contacting may be performed in a closed container, for example an air-tight and/or liquid-tight sealed container. The contacting may be performed when the growth stage of the host cell is in an exponential phase or a stationary phase. The culturing may be performed under aerobic or anaerobic conditions. The contacting may be performed under conditions where the host cell may survive in the closed container for an extended period (e.g., one or more days, weeks, or months). The conditions appropriate for the survival of the host cell may include conditions where the host cell may proliferate or may be allowed to remain in a resting state. The conditions may include providing a medium, oxygen amount, agitation and temperature that is suitable for the growth of the host cell. The medium may contain a carbon source, nitrogen source and/or other nutrients.

The contacting may include passive contacting and/or active contacting. The term ‘passive contacting’ refers to a contacting without an external driving force, and the term ‘active contacting’ refers to a contacting with an external driving force. The contacting may be achieved in a manner such that the fluorine-containing compound is injected in the form of bubbles into a solution or culture containing the host cell, or is sprayed on a cell culture. In some embodiments, the contacting may be achieved by injecting or blowing the sample into a medium or a culture broth, such as by injecting or blowing the sample from or into the bottom of the medium or the culture broth and flowing the sample to the top of the medium or culture broth. The injecting of the sample may cause the sample to break apart into smaller sizes, such as droplets in case of a liquid sample, in order to increase surface area. The contacting may be performed in a batch or continuous manner. The contacting may be performed repeatedly, such as two or more times, for example, three times, five times, or ten times or more. The contacting may be continued or repeated until the fluorine-containing compound is reduced to a desired concentration.

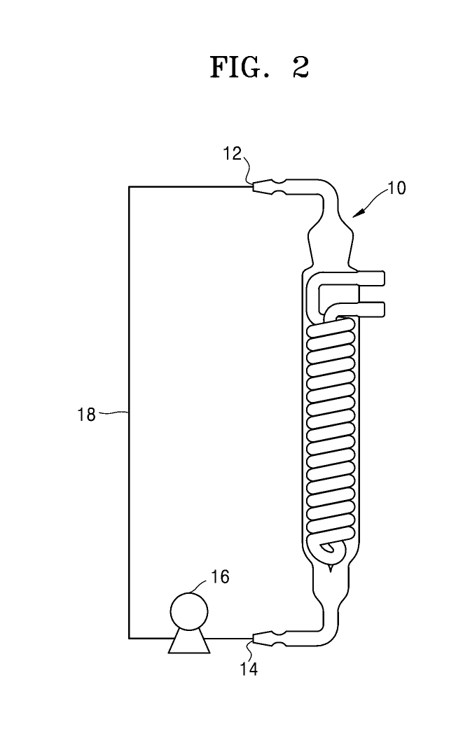

In some embodiments, the host cell may be in the form of a thin film layer, such as a liquid thin film layer. The fluorine-containing compound or sample comprising same may be in the form of a gaseous thin film layer. The liquid thin film layer, which is formed by the host cell, and the gaseous thin film layer, which is formed by the fluorine-containing compound, may contact each other. The liquid thin film layer and the gaseous thin film layer may be created by circulating a liquid in a hollow pipe and contacting the gaseous sample with the circulating liquid. The hollow pipe may be a Dimroth coiled reflux condenser shown in FIG. 2.

In an embodiment of the method, the host cell may be circulated in the sample, or the sample may be circulated in a composition (e.g., culture medium) comprising the host cell. Through the circulation process, the host cell may have an increased area or an increased period of time of contact with the fluorine-containing compound or sample comprising same. Through the circulation process, the mass transfer coefficient (KLa) value may increase, and the decomposition rate of the fluorine-containing compound may also increase.

In the method according to one or more embodiments, the sample may be an exhaust gas comprising the fluorine-containing compound. Also, contacting of the sample with the exhaust gas may further include using an exhaust gas decomposition device. The device may include one or more reactors, wherein each reactor includes at least one inlet and at least one outlet. Such a method can involve, for instance, injecting the sample into the exhaust gas decomposition device through at least one of the inlets and injecting a microorganism into the device through the at least one of the inlets. The sample and microorganism can be introduced through either the same or different inlets. Upon introduction through the inlets, the microorganism is contacted with the sample, and the concentration of the fluorine-containing compound in the sample is reduced. The resulting exhaust gas with a reduced concentration of the fluorine-containing compound (treated exhaust gas), or mixture thereof with the microorganism, may then be discharged through the one or more outlets. Again, the treated exhaust gas and microorganism can be discharged through the same or different outlets.

In the method according to one or more embodiments, the exhaust gas decomposition device may include at least a first inlet and a second inlet and at least a first outlet and a second outlet, wherein the microorganism is introduced into the device through a first inlet and discharged through a first outlet, and the sample may be introduced through the second inlet and discharged through the second outlet. In such a configuration, the microorganism may move in a direction opposite to a direction in which the sample moves, for instance, by supplying the microorganism through a different inlet and discharging from a different outlet than the sample. The microorganism may be in the form of a fluid thin film on an inner wall of the one or more reactors.

In the method according to one or more embodiments, the exhaust gas decomposition device may further include a first circulation line for re-supplying at least a portion of a fluid to the at least one first inlet, wherein the fluid contains the microorganism discharged through the first outlet. The sample including the fluorine-containing compound may remain inside the one or more reactors, or may be circulated. In addition, the one or more reactors of the exhaust gas decomposition device may further include a second inlet and a second outlet, wherein the sample may be supplied into the one or more reactors through the second inlet and discharged to the outside of the one or more reactors through the second outlet. The sample may, then, move in a second direction within the one or more reactors, wherein the second direction may be different from, for example, opposite to, the direction in which the microorganism moves. In addition, in at least one of a fluid collection zone at the inner bottom of the one or more reactors and a fluid reaction zone at the inner top of the one or more reactors of the exhaust gas decomposition device, the fluid including the microorganism and the sample including the fluorine-containing compound may contact each other, thereby decomposing the fluorine-containing compound. In the fluid reaction zone, a fluid thin film including the fluid containing the microorganism may contact a fluid including the sample.

In the method according to one or more embodiments, the exhaust gas decomposition device may further include a structure inside the one or more reactors, wherein the structure may be configured to increase a contact area between the fluid including the microorganism and the sample including the fluorine-containing compound. Any structure configured to increase a contact area between the fluid including the microorganism and the sample including the fluorine-containing compound may be included. For example, the structure may comprise a packing material or a reflux tube, but is not limited thereto. The ‘packing material’ may be inert solid material. The packing material may have various shapes. The packing material may be the same material used in the packing of a packed bed tower. The packing material may be made of plastic, magnetic material, steel or aluminium. The packing material may have very thin thickness. The packing material may have a ring shape such as rashing ring, pall ring, and berl saddle, a saddle type, and protrusion type. The packing material may be irregularly packed in the packed bed reactor. The packing material may efficiently increase contact between the fluorine-containing compound with a microorganism present in a liquid. The time or opportunity to contact the fluorine-containing compound with a microorganism can be maximized by forming a thin film of the microorganism on the surface of the packing material as well as on the inner surface of the reactor. In addition, the at least one first inlet may be connected to the fluid reaction zone at the inner top of the one or more reactors in the exhaust gas decomposition device, to thereby supply the fluid including the microorganism through the at least one first inlet.

In the method according to one or more embodiments, the fluid including the microorganism may be collected in the fluid collection zone at the inner bottom of the one or more reactors in the exhaust gas decomposition device. The sample including the fluorine-containing compound supplied into the one or more reactors through the second inlet may pass through, in the form of bubbles, the collected fluid including the microorganism to be transferred to the fluid reaction zone at the inner top of the one or more reactors, and then, may be discharged to the outside of the one or more reactors through the second outlet.

In the exhaust gas decomposition device according to certain embodiments, the aspect ratio (H/D) of the height H to the diameter D of the one or more reactors may be 2 or greater, 5 or greater, 10 or greater, 15 or greater, 20 or greater, or 50 or greater.

In the method according to one or more embodiments, the exhaust gas decomposition device may be arranged in a manner such that the side-wall of the one or more reactors, or some other internal surface thereof, is tilted or inclined at an angle in a range of about 30° to less than 90° (or greater than 90° to about 150°), about 70° to less than 90° (or greater than 90° to about110°), about 80° to less than 90° (or greater than 90° to about100°), or about 50° to less than 90°, with respect to the surface of the earth.

In the method according to one or more embodiments, the one or more reactors in the exhaust gas decomposition device may rotate. The fluid including the microorganism may be a liquid, and the sample including the fluorine-containing compound may be a gas.

The linked polynucleotide according to any of the above-described embodiments may be used to express dehalogenase on the surface of a cell.

The microorganism according to any of the above-described embodiments may be used to reduce a concentration of a fluorine-containing compound in a sample.

The method of reducing a concentration of a fluorine-containing compound in a sample, according to any of the above-described embodiments, may efficiently remove the fluorine-containing compound from the sample (e.g., by having an increased decomposition rate of the fluorine-containing compound).

One or more embodiments of the present invention will now be described in detail with reference to the following examples. However, these examples are only for illustrative purposes and are not intended to limit the scope of the invention.

### EXAMPLE 1

### Haloacid Dehalogenase-Cell Surface Displaying Microorganism and Removal of Fluorine-Containing Compound by Using the Microorganism

In the present example, the haloacid dehalogenase gene SF0757 was amplified from the Bacillus bombysepticus SF3 strain, and then ligated with an anchoring motif to express the gene on the cell surface. The detailed processes were as follows.

1. Preparation of Polynucleotide that Encodes Anchoring Motif

An N-terminal domain of a Bacillus anthracis—derived exosporium protein (BclA: NCBI Accession No. CAD56878.1) was used as the anchoring motif. BclA had an amino acid sequence of SEQ ID NO: 11 and was encoded by a nucleotide sequence of SEQ ID NO: 12.

The BclA contains a 19-residue amino terminal peptide, which is proteolytically removed during sporulation, and the remaining mature BlcA is attached to the surface of a developing forespore. The mature BclA protein consists of three domains: an N-terminal domain (NTD), a C-terminal domain (CTD), and a central domain. The central domain contains 1 to 8 repeating regions of a ‘(GPT)xGDTGTT triplet sequence.’ The central domain (also called “collagen-like protein” or “CLR”) resembles a mammalian collagen protein. According to the repeating number of CLR, the BclA may have different sizes. To develop an effective surface display system, different motifs (BAN and BANF) were used. The BAN contains only 21 amino acids (SEQ ID NO: 13) consisting of the amino acid corresponding to position 20 through the amino acid corresponding to position 40, without the first 19-residue amino-terminal peptide of BclA. The BANF contains 40 amino acids (SEQ ID NO: 15) consisting of the amino acid corresponding to position 1 through the amino acid corresponding to position 40. The haloacid dehalogenase was fused to the C-terminus of each anchoring motif.

First, polynucleotides (SEQ ID NO: 14 and SEQ ID NO: 16, respectively) encoding respective BAN and BANF polypeptides were synthesized (Cosmo Gentech Co., Ltd., Korea).

2. Construction of Recombinant Strain Expressing Anchoring Motif and Haloacid Dehalogenase (SF0757) Fusion Gene

In the present example, the Bacillus bombysepticus SF3 strain capable of reducing a concentration of CF₄ in waste water discharged from a semiconductor factory was screened.

Sludge in waste water discharged from a Samsung Electronics plant (Giheung, Korea) was smeared on an agar plate including a carbon-free medium (containing 0.7 g/L of K₂HPO₄, 0.7 g/L of MgSO₄.7H₂O, 0.5 g/L of (NH₄)₂SO₄, 0.5 g/L of NaNO₃, 0.005 g/L of NaCl, 0.002 g/L of FeSO₄.7H₂O, 0.002 g/L of ZnSO₄.7H₂O, 0.001 g/L of MnSO₄, and 15 g/L of Agar), and the agar plate was put in a GasPak™ Jar (available from BD Medical Technology). The jar was filled with 99.9 v/v % of CF₄, sealed, and then statically cultured at a temperature of 30° C. under anaerobic conditions. After the culturing, single colonies formed on the agar plate were further cultured using a high throughput screening (HTS) system (Thermo Scientific/Liconic/Perkin Elmer). Each of the cultured single colonies was then inoculated on a 96-well microplate, wherein each well contained 100 μL of LB medium. The LB medium used included 10 g/L of tryptone, 5 g/L of yeast extract, and 10 g/L of NaCl. While the 96-well microplate was statically cultured at a temperature of about 30° C. under aerobic conditions for 96 hours, the growth of the single colonies was observed by measuring absorbance at 600 nm every 12 hours.

The top 2% of strains showing excellent growth were selected and then inoculated in a glass serum bottle (having a volume of 75 mL) containing 10 mL of the LB medium to reach an OD₆₀₀ of 0.5. The glass serum bottle was sealed, and CF₄ was injected thereto using a syringe to a concentration of 1,000 ppm. The glass serum bottle was then incubated in a shaking incubator at a temperature of 30° C. for 4 days while being stirred at a speed of 230 rpm. Then, an amount of CF₄ in a headspace of the glass serum bottle was analyzed.

0.5 mL of the gas in the headspace was sampled using a syringe to analyze the amount of CF₄ under the same conditions as described in Section 3, below. For a control group, 1,000 ppm of a cell-free CF₄ gas was analyzed after incubation under the same conditions.

As a result, the concentration of CF₄ in a separated microorganism among the tested strains was reduced by 10.27%, compared to the cell-free control group. The microorganism exhibited a decomposition activity of 0.02586 g/kg-cell. To identify the selected strain, genome sequences thereof were analyzed.

A genome obtained by assembling three contigs by next generation sequencing (NGS) had a final size of 5.3 Mb, and as a result of gene annotation, was found to contain 5,490 genes in total. As a result of phylogenetic tree analysis of each contig, the microorganism was found to belong to the genus Bacillus bombysepticus.

The isolated microorganism was named Bacillus bombysepticus SF3, deposited at the Korean Collection for Type Cultures (KCTC), an international depository authority under the Budapest Treaty, on Feb. 24, 2017, with Accession No. KCTC 13220BP.

Through genomic sequence analysis of the Bacillus bombysepticus SF3 strain identified above, gene SF0757 (SEQ ID NO: 10), assumed to encode dehalogenase, was selected.

After the B. bombysepticus SF3 strain was cultured overnight in an LB medium at a temperature of 30° C. while being stirred at a speed of 230 rpm, genomic DNA thereof was isolated using a total DNA extraction kit (Invitrogen Biotechnology). In order to obtain and amplify the gene SF0757, PCR was performed using the genomic DNA as a template and oligonucleotides having SEQ ID NOS: 17 and 18 as a primer set. The amplified gene SF0757 was ligated with pTrc99A (Pharmacia Biotech, Uppsala, Sweden) (4.17 kb, bla, trc promoter), which was cleaved with restriction enzymes Ncol and Xbal, using an InFusion Cloning Kit (Clontech Laboratories, Inc.) to construct a pTrc-SF0757 vector (Control group).

In the presence of the polynucleotide encoding the BANF polypeptide as synthesized above in Section 1, PCR was performed using oligonucleotides of SEQ ID NOs: 19 and 20 as a primer set to obtain and amplify a corresponding BANF DNA fragment. PCR was also performed using the genomic DNA of the B. bombysepticus SF3 strain as a template and oligonucleotides of SEQ ID NOs: 21 and 18 as a primer set to amplify a SF0757 DNA fragment. PCR was then performed using the amplified BANF DNA fragment and SF0757 DNA fragment as templates and oligonucleotides of SEQ ID NOs: 19 and 18 as a primer set to obtain a BANF-SF0757 fusion gene. The BANF-SF0757 fusion gene thus obtained was ligated with pTrc99A (Pharmacia Biotech, Uppsala, Sweden) (4.17 kb, bla, trc promoter), which was cleaved with restriction enzymes Ncol and Xbal, using an InFusion Cloning Kit (Clontech Laboratories, Inc.), to construct a pTrc-BANF-SF0757 vector.

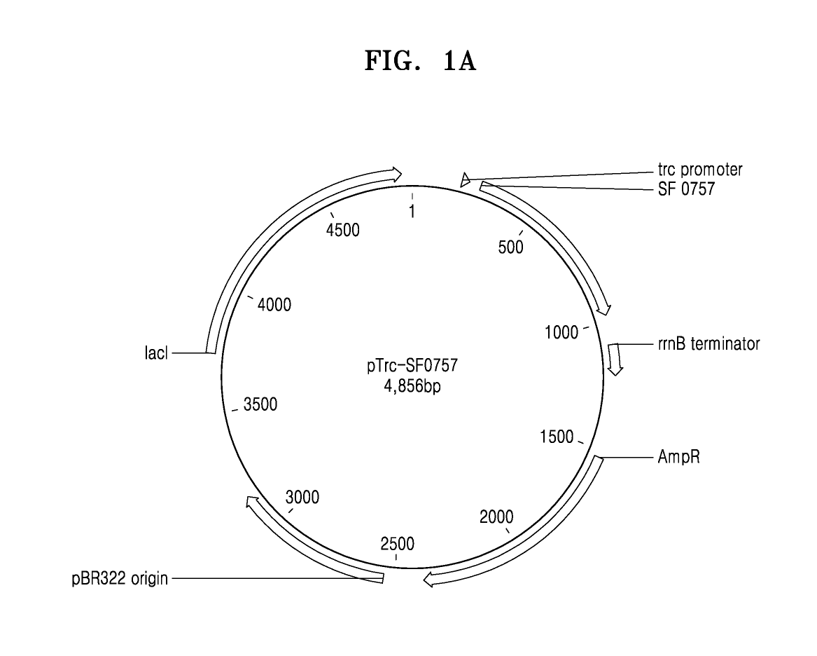

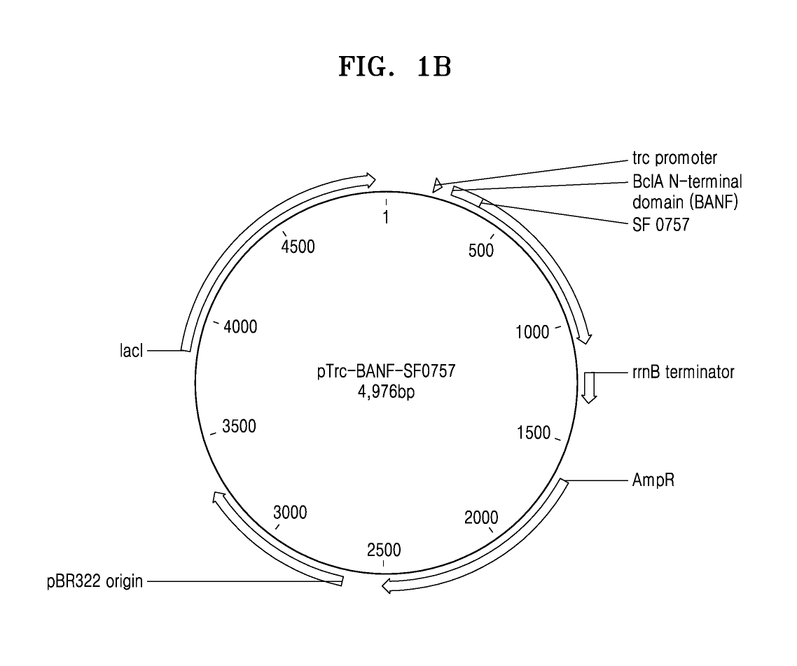

FIG. 1A is a vector map of the pTrc-SF0757 vector.

FIG. 1B is a vector map of the pTrc-BANF-SF0757 vector.

Next, the constructed pTrc-BANF-SF0757 and pTrc-SF0757 vectors were each introduced into E. coli W3100 by a heat shock method, and then cultured in an LB plate medium containing 100 μg/mL of ampicillin to select strains having ampicillin resistance. The selected strain was named recombinant Escherichia coli (E. coli) W3110/pTrc-BANF-SF0757. This strain was labeled with an RFP fluorescent tag to measure fluorescence in the outer cell membrane, and as a result, expression of the fusion gene was found on the cell surface.

3. Decomposition of Fluorine-Containing Compound CF₄ by Using Circulation Process

The CF₄ removal effects of the recombinant E. coli W3110/pTrc-SF0757 strain (Control group) and the E. coli W3110/pTrc-BANF-SF0757 strain, into which the SF0757 and BANF-SF0757 genes constructed above in Section 2 were introduced, respectively, were comparatively evaluated using a circulation process.

In particular, the recombinant E. coli W3110/pTrc-SF0757 and W3110/pTrc-BANF-SF0757 strains were each incubated in a LB medium at about 30° C. while being stirred at about 230 rpm to reach an OD₆₀₀ of about 0.5. After 0.2 mM of IPTG was added thereto, each culture medium was incubated overnight at about 20° C. while being stirred at about 230 rpm.

After 50 mL of a M9 medium (containing 6 g of Na₂HPO₄, 3 g of KH₂PO₄, 0.5 g of NaCl, and 1 g of NH₄Cl per 1 L of distilled water) and 1,000 ppm of CH₄ gas were added to a glass Dimroth coiled reflux condenser (a reactor length: 350 mm, an exterior diameter: 35 mm, and an interior volume: 200 mL) that was sterilized and vertically oriented, as shown in FIG. 2, the M9 medium was circulated. FIG. 2 is a schematic view of the glass Dimroth coiled reflux condenser (10). The M9 medium was supplied through an inlet (12) at an upper portion of the condenser (10), flowed through an inner wall of the condenser (10), and then discharged through an outlet (14) of a lower portion of the condenser (10). The discharged M9 medium was re-supplied into the inlet (12) along a circulation line (18). Although not shown in FIG. 2, to maintain a temperature of the condenser (10), an inner screwed pipe of the condenser (10) was connected to a constant-temperature bath having a temperature of 30° C. The circulation is performed by a pump (16). Here, the circulation rate of the M9 medium was maintained at about 4 mL/min. After a predetermined time of about 144 hours, the amount of CF₄ gas in the condenser was measured by gas chromatography-mass spectrometry (GC-MS). As a result, there was no change in the amount of CF₄ gas in the condenser.

Next, the recombinant microorganisms were inoculated and suspended in an M9 medium by using a syringe, to reach an initial concentration at OD₆₀₀ of 5.0. The recombinant microorganisms were the recombinant E. coli to which the BANF-SF0757 and SF0757 genes were introduced, respectively. CF₄ decomposition rates of the recombinant E. coli strains to which the SF0757 gene (Control group) and the BANF-SF0757 gene were respectively introduced were compared. E. coli to which an empty vector was introduced was used as a negative control group. There was no change in CF₄ level in the negative control group.

The circulation rate of the M9 culture medium was about 4 mL/min, and the temperature inside the condenser was maintained at 30° C. After 144 hours from the inoculation of the strain, the amount of CF₄ gas in the condenser was measured by GC-MS. A decomposition rate of the CF₄ gas was calculated using Equation 1. The results are shown in Table 1.

Decomposition rate of CF₄=[(Initial amount of CF₄−Amount of CF₄ after reaction)/Initial amount of CF₄]×100 <Equation 1>

TABLE 1

| Strain | CF₄ decomposition rate (%) |
|---|---|
| W3110/pTrc-SF0757 | 21.8 |
| W3110/pTrc-BANF-SF0757 | 34.5 |

Referring to Table 1, after incubation for about 144 hours, the recombinant strain W3110/pTrc-BANF-SF0757 expressing SF0757 on its cell surface was found to exhibit a CF₄ decomposition rate about 1.6 times higher than the recombinant strain W3100/pTrc-SF0757 expressing SF0757 inside cells, when a gas circulation process was used.

### EXAMPLE 2

### Fluoroacetate Dehalogenase-Cell Surface Display Microorganism and Removal of Fluorine-Containing Compound by Using the Microorganism

In the present example, the fluoroacetate dehalogenase gene FAcD from the Burkholderia sp. FA1 strain was synthesized, and then ligated with an anchoring motif to express the gene on a cell surface. Detailed processes were as follows.

1. Construction of Anchoring Motif and Fluoroacetate Dehalogenase (FAcD) Fusion Gene Expression Recombinant E. coli Strain

As described above in Section 1 of Example 1, polynucleotides encoding the respective anchoring motif BAN and BANF polypeptides, and FAcD gene (SEQ ID NO: 8) were synthesized (Cosmo Gentech Co., Ltd., Korea).

To amplify the FAcD gene, PCR was performed using the synthesized FAcD as a template and oligonucleotides of SEQ ID NOs: 22 and 23s as a primer set.

The amplified FAcD gene was ligated with pTrc99A, which was cleaved with restriction enzymes Ncol and Xbal, using an InFusion Cloning Kit (Clontech Laboratories, Inc.) to construct a pTrc-FAcD vector (Control group).

PCR was performed using the synthesized polynucleotide that encodes the BANF polypeptide as a template and oligonucleotides of SEQ ID NOs: 19 and 24 as a primer set to amplify a corresponding BANF DNA fragment. PCR was also performed using the synthesized FAcD as a template and oligonucleotides of SEQ ID NOs: 25 and 23 as a primer set to amplify a corresponding FAcD DNA fragment. PCR was then performed using the amplified BANF DNA fragment and FAcD DNA fragment as templates and oligonucleotides of SEQ ID NOs: 19 and 23 as a primer set to obtain a BANF-FAcD fusion gene. The BANF-FAcD fusion gene thus obtained was ligated with pTrc99A, which was cleaved with restriction enzymes Ncol and Xbal, using an InFusion Cloning Kit (Clontech Laboratories, Inc.), to construct a pTrc-BANF-FAcD vector.

PCR was also performed using the synthesized polynucleotide that encodes the BAN polypeptide as a template and oligonucleotides of SEQ ID NOs: 26 and 24 as a primer set to amplify a corresponding BAN DNA fragment. PCR was further performed using the synthesized FAcD as a template and oligonucleotides of SEQ ID NOs: 25 and 23 as a primer set to amplify a corresponding FAcD DNA fragment. PCR was then performed using the amplified BAN DNA fragment and FAcD DNA fragment as templates and oligonucleotides of SEQ ID NOs: 26 and 23 as a primer set to obtain a BAN-FAcD fusion gene. The BAN-FAcD fusion gene thus obtained was ligated with pTrc99A, which was cleaved with restriction enzymes Ncol and Xbal, using an InFusion Cloning Kit (Clontech Laboratories, Inc.), to construct a pTrc-BAN-FAcD vector.

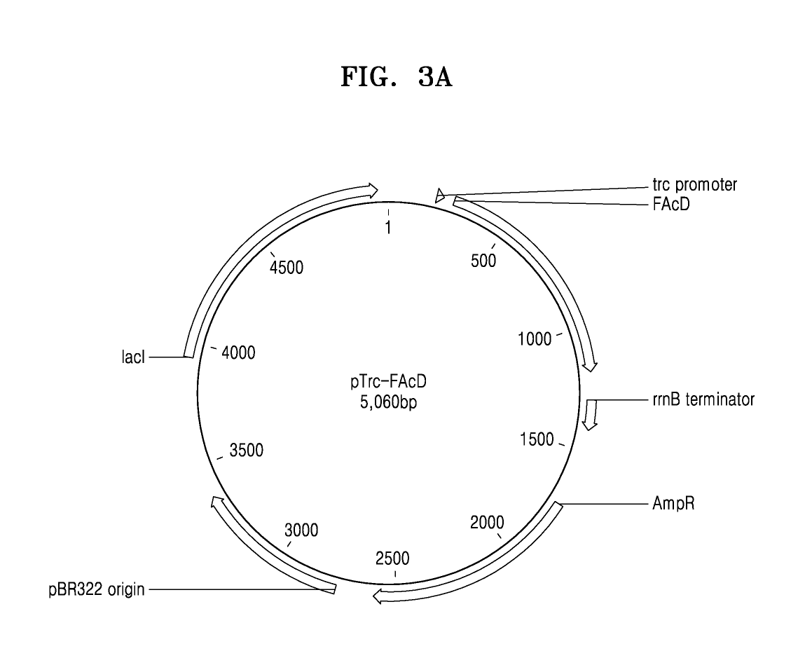

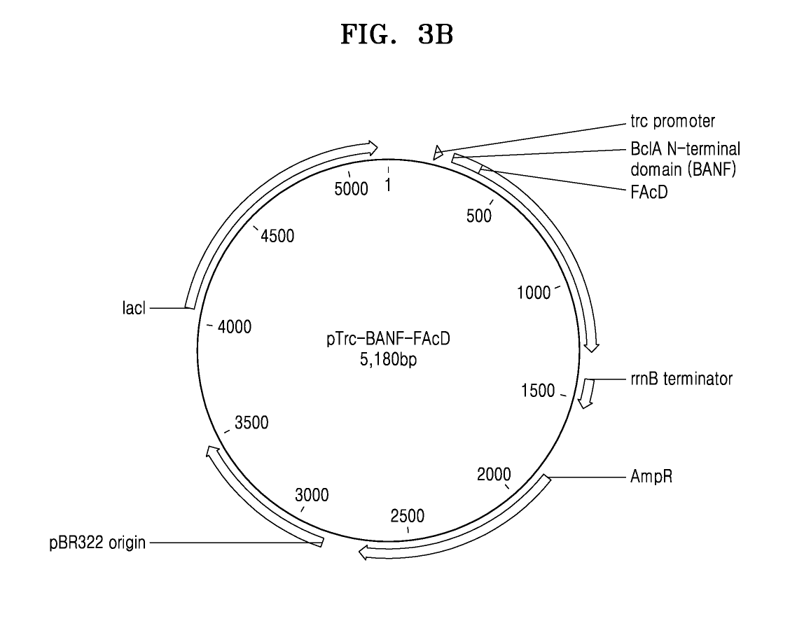

FIG. 3A is a vector map of the pTrc-FAcD vector.

FIG. 3B is a vector map of the pTrc-BANF-FAcD vector.

The FAcD has a nucleotide sequence of SEQ ID NO: 8, which encodes an amino acid sequence of SEQ ID NO: 7.

Next, the constructed pTrc-FAcD, pTrc-BAN-FAcD, and pTrc-BANF-FAcD vectors were each introduced to E. coli W3110 by a heat shock method, and then cultured in an LB plate medium containing 100 μg/mL of ampicillin to select strains having ampicillin resistance. The selected strains were named recombinant E. coli W3110/pTrc-FAcD, W3110/pTrc-BAN-FAcD, and W3110/pTrc-BANF-FAcD, respectively. These strains were labeled with an RFP fluorescent tag to measure fluorescence in the outer cell membrane, and as a result, expression of the fused protein was found on the cell surface of W3110/pTrc-BAN-FAcD and W3110/pTrc-BANF-FAcD.

2. Effect of FAcD-Surface-Displaying Recombinant E. coli on Removal of CFH₂COOH in Sample

The fluoroacetate (FA) removal effects of the recombinant E. coli W3110/pTrc-FAcD (Control group), W3110/pTrc-BAN-FAcD, and W3110/pTrc-BANF-FAcD strains, into which the FAcD, BAN-FAcD, and BANF-FAcD genes constructed above in Section 1 were introduced, respectively, in a sample, were comparatively evaluated.

In particular, the recombinant E. coli W3110/pTrc-FAcD, W3110/pTrc-BAN-FAcD, and W3110/pTrc-BANF-FAcD strains were each incubated in an LB medium at about 30° C. while being stirred at about 230 rpm to reach an OD₆₀₀ of about 0.5. After 0.2 mM of IPTG was added thereto, each culture medium was incubated overnight at about 20° C. while being stirred at about 230 rpm. The cultured cells were collected and then suspended in an M9 medium to reach a cell concentration at OD₆₀₀ of about 3.0. 10 mL of the cell solution was added to a 60-mL serum bottle, and the serum bottle was sealed. Next, liquid fluoroacetate (FA) was injected using a syringe through a rubber cap stopper of the serum bottle to a concentration of about 9.5 mM, and then the serum bottle was incubated at about 30° C. for about 30 minutes while being stirred at about 230 rpm. This experiment was done in triplicate. After the incubation, fluorine ions (F⁻) in the culture medium (supernatant) contained in the serum bottle were detected using a fluorine selective electrode (perfectION™, Mettler, Toledo, Switzerland), and a concentration of the fluorine ions were quantized using a SevenMulti™ (Mettler Toledo, Switzerland), based on the calibration curve created using fluorine standard solutions (0.04, 0.08, 0.20, 0.58, and 0.96 mg/L). A reagent used in the analysis was prepared by adding 5 mL of the culture medium to 5 mL of a low-level TISAB solution. The low-level TISAB solution was prepared by dissolving 57 mL of acetic acid and 58 g of sodium chloride in 1 L of water and adjusting pH to 5.3 with a 5M NaOH solution.

The FAcD is known to catalyze the conversion of CFH₂FCOO⁻+H₂O to CH₂(OH)COO⁻+H⁺+F⁻. Accordingly, a concentration of fluorine ions in a test sample indicates a degree of decomposition of the fluoroacetate.

Table 2 represents the FAcD-surface display recombinant strains' activities of removing fluoroacetate in a sample, relative to the fluoroacetate removal activity of the wild type.

TABLE 2

| Strain | Fluorine ion (mg/L) |
|---|---|
| Control group | 1.2 |
| W3110/pTrc-FAcD | 8.9 |
| W3110/pTrc-BAN-FAcD | 15.0 |
| W3110/pTrc-BANF-FAcD | 99.1 |

In Table 2, the control group refers to E. coli W3110 to which an empty pTrc99A vector was introduced.

Referring to Table 2, the recombinant strains W3110/pTrc-BAN-FAcD and W3110/pTrc-BANF-FAcD expressing FAcD on the cell surface were found to have a fluorine concentration in the sample about 1.7 times and 11.1 times higher, respectively, than the W3110/pTrc-FAcD strain expressing FAcD in the cells, indicating that the cells expressing FAcD on the cell surface have an improved fluoroacetate decomposition rate.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A linked polynucleotide comprising a first polynucleotide, a second polynucleotide, and a third polynucleotide that are operatively linked to one another, wherein the first polynucleotide is a promoter polynucleotide, the second polynucleotide is a polynucleotide encoding an anchoring motif, and the third polynucleotide is a polynucleotide encoding a dehalogenase.

2. The polynucleotide of claim 1, wherein the linked polynucleotide is constructed such that, when expressed in a host cell, a fusion protein of the anchoring motif and the dehalogenase is expressed on the surface of the host cell.

3. The polynucleotide of claim 2, wherein the anchoring motif comprises a transmembrane portion and a linker portion, wherein the linker portion connects the transmembrane portion and the dehalogenase.

4. The polynucleotide of claim 1, wherein the anchoring motif is selected from the group consisting of a membrane protein, a lipoprotein, and an autotransporter protein.

5. The polynucleotide of claim 1, wherein the anchoring motif is selected from the group consisting of BclA of Bacillus; OmpA, Lpp-OmpA, OmpC, OmpS, LamB, OmpC, Lpp-OmpC, PhoE, and FadL of Escherichia coli (E. coli); OmpC of Salmonella; OprF of Pseudomonas; adhesin involved in diffusion adherence (AIDA-I) of pathogenic E. coli; and fragments thereof.

6. The polynucleotide of claim 1, wherein the anchoring motif is BclA comprising SEQ ID NO: 1, SEQ ID NO: 3, or SEQ ID NO: 5, or a fragment thereof.

7. The polynucleotide of claim 1, wherein the dehalogenase belongs to EC 3.8.1.3 or EC 3.8.1.2.

8. The polynucleotide of claim 1, wherein the dehalogenase has about 85% or greater sequence identity to SEQ ID NO: 7 or SEQ ID NO: 9.

9. A host cell comprising the linked polynucleotide of claim 1 and expresses a dehalogensase on the cell surface.

10. The host cell of claim 9, wherein the host cell expresses a fusion protein comprising the anchoring motif and the dehalogenase, and wherein the dehalogenase is expressed on a surface of the host cell.

11. The host cell of claim 9, wherein the anchoring motif comprises a transmembrane portion and a linker portion, wherein the linker portion connects the transmembrane portion to the dehalogenase.

12. The host cell of claim 9, wherein the anchoring motif is selected from the group consisting of a membrane protein, a lipoprotein, and an autotransporter protein.

13. The host cell of claim 9, wherein the anchoring motif is selected from the group consisting of BclA of Bacillus; OmpA, Lpp-OmpA, OmpC, OmpS, LamB, OmpC, Lpp-OmpC, PhoE, and FadL of Escherichia coli (E. coli); OmpC of Salmonella; OprF of Pseudomonas; adhesin involved in diffusion adherence (AIDA-I) of pathogenic E. coli; and fragments thereof.

14. The host cell of claim 9, wherein the anchoring motif is BclA comprising SEQ ID NO: 1, SEQ ID NO: 3, or SEQ ID NO: 5, or a fragment thereof.

15. The host cell of claim 9, wherein the dehalogenase belongs to EC 3.8.1.3 or EC 3.8.1.2.

16. The host cell of claim 9, wherein the dehalogenase has about 85% or greater sequence identity to SEQ ID NO: 7 or SEQ ID NO: 9.

17. A method of reducing a concentration of a fluorine-containing compound in a sample, the method comprising contacting a sample comprising a fluorine-containing compound with the host cell of claim 9 to reduce the concentration of the fluorine-containing compound in the sample, wherein the fluorine-containing compound is represented by Formula 1, Formula 2, or Formula 3:
C(R₁)(R₂)(R₃)(R₄) <Formula 1>
(R₅)(R₆)(R₇)C—[C(R₁₁)(R₁₂)]ₙ—C(R₈)(R₉)(R₁₀) <Formula 2>
(R₁₃)(R₁₄)(R₁₅)C—[C(R₁₉)(R₂₀)]ₙ-A, <Formula 3>
wherein, in Formulae 1, 2, and 3, n is an integer from 0 to 10;
when n in Formula 2 is 2 or greater, each R₁₁ is the same or different from one another, and each R₁₂ is the same or different from one another;
when n in Formula 3 is 2 or greater, each R₁₉ is the same or different from one another, and each R₂₀ is the same or different from one another,
A is —COOH or —C(R₁₆)(R₁₇)(R₁₈),
R₁, R₂, R₃, and R₄ are each independently fluorine (F), chlorine (Cl), bromine (Br), iodine (I), or hydrogen (H), wherein at least one of R₁, R₂, R₃, and R₄ is F;
R₅, R₆, R₇, R₈, R₉, R₁₀, R₁₁, and R₁₂ are each independently F, Cl, Br, I, or H, wherein at least one of R₅, R₆, R₇, R₈, R₉, R₁₀, R₁₁, and R₁₂ is F;
R₁₃, R₁₄, R₁₅, R₁₆, R₁₇, R₁₈, R₁₉, and R₂₀ are each independently F, Cl, Br, I, H, or —COOH, wherein at least one of R₁₃, R₁₄, R₁₅, R₁₆, R₁₇, R₁₈, R₁₉, and R₂₀ is F, and wherein at least one of R₁₃, R₁₄, R₁₅, R₁₆, R₁₇, R₁₈, R₁₉, and R₂₀ is —COOH.

18. The method of claim 17, wherein the anchoring motif is BclA comprising SEQ ID NO: 1, SEQ ID NO: 3, or SEQ ID NO: 5, or a fragment thereof.

19. The method of claim 17, wherein the dehalogenase belongs to EC 3.8.1.3 or EC 3.8.1.2.

20. The method of claim 17, wherein the dehalogenase has about 85% or greater sequence identity to SEQ ID NO: 7 or SEQ ID NO: 9.

21. The method of claim 17, wherein the host cell further comprises a dehalogenase-encoding polynucleotide that is not linked to a polynucleotide encoding the anchoring motif, and the host cell simultaneously expresses a dehalogenase on the cell surface and a dehalogenase that remains inside the cell.

22. The polynucleotide of claim 1, further comprising a fourth polynucleotide encoding a signal polypeptide linked between the first and second polynucleotides.

23. The method of claim 17, wherein the contacting occurs in an exhaust gas decomposition device comprising one or more reactors, each of which comprises at least one first inlet and a first outlet.

24. The method of claim 23, wherein the contacting comprises injecting the sample into the exhaust gas decomposition device; and injecting the host cell into the device through the at least one first inlet, so that the host cell contacts the sample and the resulting mixture is discharged through the first outlet.

25. A method of preparing a recombinant cell that expresses dehalogenase on the surface of the cell, the method comprising introducing into the cell a polynucleotide of claim 1.
